(19) United States
(12) Patent Application Publication
(10) Pub. No.: US 2026/0365855 A1
(43) Pub. Date: Nov. 19, 2026

(54) TARGETED QUINOCYANINE DYES FOR THE INTRAOPERATIVE DELINEATION OF CANCER MARGINS

(71) Applicant: THE TRUSTEES OF THE UNIVERSITY OF PENNSYLVANIA, Philadelphia, PA (US)

(72) Inventors: Edward J. DELIKATNY, Havertown, PA (US); Michael C. HART, Philadelphia, PA (US); Anatoliy V. POPOV, Bryn Mawr, PA (US); Isuri RITESH, Philadelphia, PA (US)

(21) Appl. No.: 19/119,823

(22) PCT Filed: Oct. 10, 2023
(86) PCT No.: PCT/US2023/076452

§ 371 Date: Apr. 10, 2025

Related U.S. Application Data
(60) Provisional application No. 63/378,985, filed on Oct. 10, 2022.

(51) Int. Cl. C09B 23/01; A61K 49/00

(52) U.S. Cl. CPC C09B 23/0066; A61K 49/0032

(57) ABSTRACT

The disclosure describes compound 1, compound 2, compound 3, compound 4, compound 5, compound 6 and compound 7: Pharmaceutical compositions comprising compound 1, compound 2, compound 3, compound 4, compound 5, compound 6 and compound 7, as well as methods of their use and preparation, are also described.

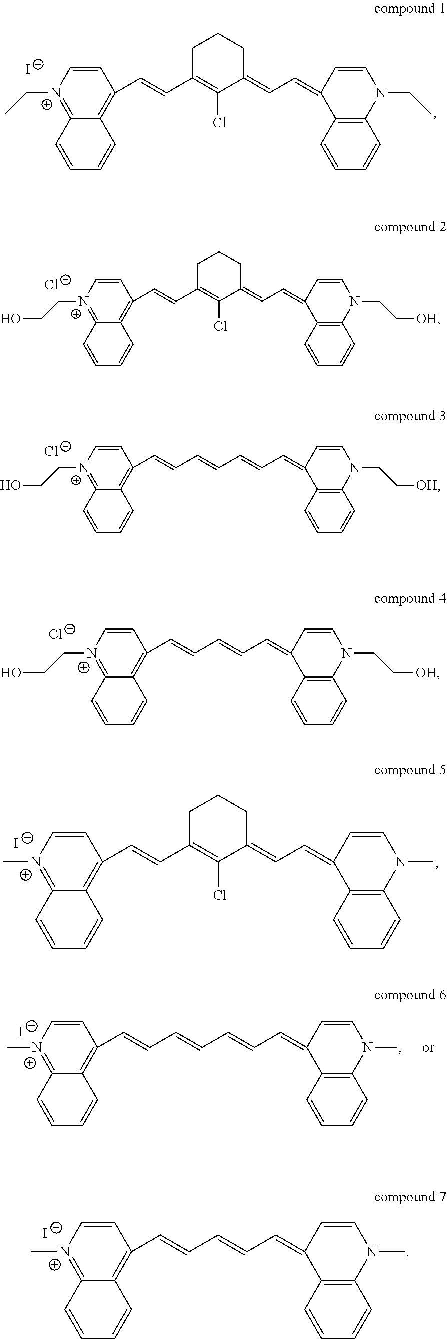

### CROSS REFERENCE TO RELATED APPLICATIONS

This application claims the benefit of U.S. Provisional Application Nos. 63/378,985, filed Oct. 10, 2022, the entirety of which is incorporated by reference herein for any and all purposes.

### GOVERNMENT RIGHTS

This invention was made with government support under CA226412 and CA201328 awarded by the National Institutes of Health. The government has certain rights in the invention.

### TECHNICAL FIELD

The present disclosure relates to the field of dyes for visualization of certain cell types, including cells present in cancer margins.

### BACKGROUND

At present, a variety of different cancer types are primarily treated through surgical resection. The nonspecific nature of delineation of tumor margins intraoperatively leads to high rates of disease recurrence. For example, the primary treatment of non-small cell lung carcinomas (NSCLC) is surgical resection relying on visual inspection and tissue palpation to identify cancerous tissue. In these cases, disease recurrence occurs in approximately forty percent of cases. Accordingly, there is a long-felt need in the art for improved fluorphores that can be used in imaging biological tissue.

### SUMMARY

By developing a fluorophore with emission in the near-infrared II (NIR-II) window, imaging through biological tissue is less hindered by tissue absorbance and scattering. Further, currently available NIR-II fluorophores exhibit either high toxicity or lack of specificity.

Currently, one of the primary treatments for these cancers is surgical resection of the tumor. The disclosed probes can fluoresce in the near-infrared II (NIR-II) window. By using the disclosed fluorescent molecules targeted to an enzyme overexpressed in cancers, one can image tumors in vivo and delineate tumor margins to improve the rate of complete tumor resections improving clinical outcomes.

Penetration of light through tissue increases with longer wavelengths. To maximize resolution, fluorophores have been developed to absorb and emit light in the near-infrared I (NIR-I) window. One of these fluorophores, indocyanine green (ICG), has recently demonstrated a measurable fluorescence at even longer wavelengths of light in the near-infrared II (NIR-II) range. Further decreased tissue scattering, absorption, and auto-fluorescence in the NIR-II range improves resolution in vivo. Due to the lack of fluorimeters able to detect NIR-II fluorescence, particularly in the life science, few fluorophores have been designed to fluoresce in the NIR-II region. Thus, innovation in the realm of NIR-II imaging has been stalled due to the high financial and time burden associated with self-engineering NIR-II imaging systems. Recently, commercially available fluorimeters have been developed to measure fluorescence in the NIR-II region designed for in vivo imaging. The disclosed technology coincides with the development of these new instruments and represent one of the first waves of NIR-II research for biomedical applications.

In one aspect, the present disclosure provides a compound having the structure of compound 1, compound 2, compound 3, compound 4, compound 5, compound 6 or compound 7:

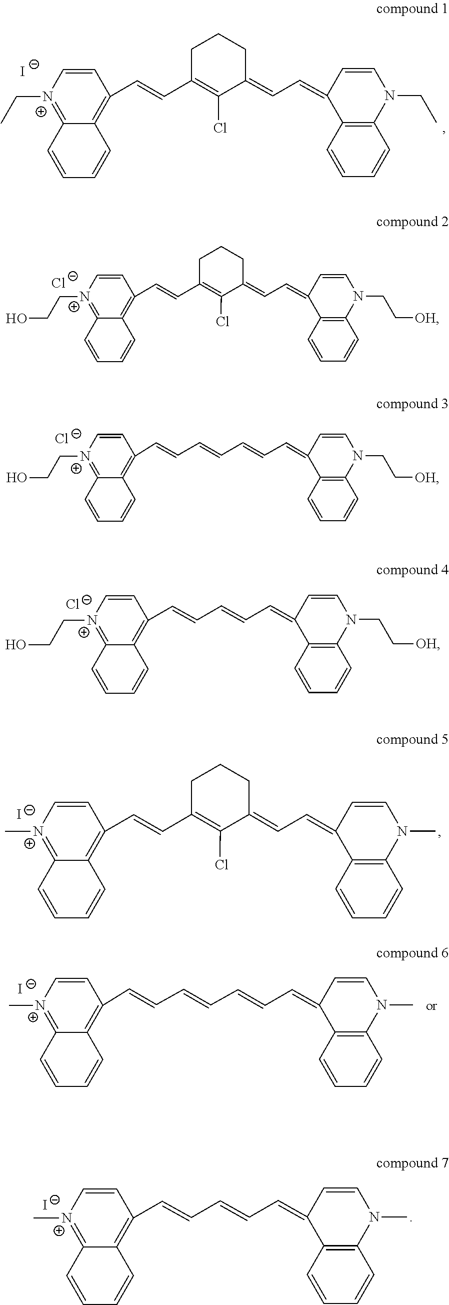

Stereoisomers of compound 1, compound 2, compound 3, compound 4, compound 5, compound 6 and compound 7, and the pharmaceutical salts thereof, are also contemplated, described, and encompassed herein. Methods of using compound 1, compound 2, compound 3, compound 4, compound 5, compound 6 and compound 7 are described, as well as pharmaceutical compositions including compound 1, compound 2, compound 3, compound 4, compound 5, compound 6 and compound 7.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 depicts absorbance and emission spectra of compound 1, compound 2 and compound 3. FIG. 1A shows absorbance and emission spectra for compound 1 and compound 2. FIG. 1B shows the maximum absorbance and maximum emission for compound 1. FIG. 1C shows emission intensities for 5 μM compound 1, compound 2 and compound 3.

FIG. 2 depicts NIR-II emissions of serial dilutions of compound 1, JAS239, and ICG.

FIG. 3 depicts NIR-II emissions of compound 1, JAS239, and ICG at varying tissue depths.

FIG. 4 depicts a NIR-II emissions of KLN 205 cell lysates following treatment with compound 1, compound 2 and compound 3, or control.

FIG. 5 depicts imaging of ChoKα NIR-I probe (JAS239) and ChoKα NIR-II probe (compound 1) using tissue phantom. FIG. 5A shows uncovered plates (left panel) or plates covered by 2 mm chicken tissue (right panel). FIG. 5B shows SBR for each fluorescent spot of the images.

FIG. 6 depicts imaging of intracellular uptake and retention of ChoKα-targeted NIR-II probe (compound 1) by KLN-205 cancer cells. FIG. 6A shows intracellular uptake for compound 1 (top panel) and intracellular uptake for the control compound (bottom panel). FIG. 6B shows RFU for compound 1 and for the control compound.

FIG. 7 depicts imaging of mice injected with compound 1. FIG. 7A shows a ventral image of mice at 5 mins after injection. FIG. 7B shows a ventral image of mice at 2 hours after injection.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. Alkyl groups of the disclosure can be unsubstituted or substituted. In those embodiments wherein the alkyl group is substituted, the alkyl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substituents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “alkoxide” refers to the conjugate base of an alcohol and includes an organic group bonded to a negatively charged oxygen atom.

The term “halo” or “halogen,” refers to chloro, fluoro, bromo, or iodo.

The term “haloalkyl” refers to any alkyl radical having one or more hydrogen atoms replaced by a halogen atom.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Cycloalkyl groups of the disclosure include monocyclic groups, as well as multicyclic groups such as bicyclic and tricyclic groups. In those embodiments having at least one multicyclic cycloalkyl group, the cyclic groups can share one common atom (i.e., spirocyclic). In other embodiments having at least one multicyclic cycloalkyl group, the cyclic groups share two common atoms. Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), spiro[3.3]heptanyl, bicyclo[3.3.0]octanyl, and the like. Cycloalkyl groups of the disclosure can be unsubstituted or substituted. In those embodiments wherein the cycloalkyl group is substituted, the cycloalkyl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substituents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “cycloalkenyl” refer to cyclic, non-aromatic hydrocarbon groups having from 3 to 10 carbon atoms (“C₃-C₁₀”), preferably from 3 to 6 carbon atoms (“C₃-C₆”) and containing at least one carbon-carbon double bond. For example, cycloalkenyl groups include, but are not limited to cyclopropenyl, cyclobutenyl, and the like.

The term “heterocycloalkyl” when used alone or as part of a substituent group refers to any three to ten membered monocyclic or bicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms. The term —C₃-C₆ heterocycloalkyl refers to a heterocycloalkyl group having between three and six carbon ring atoms. The term —C₃-C₁₀ heterocycloalkyl refers to a heterocycloalkyl group having between three and 10 ring atoms. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, and the like. Heteroycloalkyl groups of the disclosure can be unsubstituted or substituted. In those embodiments wherein the heterocycloalkyl group is substituted, the heterocycloalkyl group can be substituted with 1, 2, or 3 substituents independently selected from D, —OH, —CN, amino, halo, oxo, C₁-C₆alkyl, C₁-C₆alkoxy, C₁-C₆haloalkyl, and C₁-C₆haloalkoxy. Additional substituents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “heterocycloalkenyl” when used alone or as part of a substituent group refers to any three to ten membered monocyclic or bicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkyenyl group, the cyclic groups can share one common atom (i.e., spirocyclic). In other embodiments having at least one multicyclic heterocyclo-alkenyl group, the cyclic groups share two common atoms. The term —C₃-C₆ heterocyclo-alkenyl refers to a heterocycloalkenyl group having between three and six carbon atoms. The term —C₃-C₁₀ heterocycloalkenyl refers to a heterocycloalkenyl group having between three and ten ring atoms. The heterocycloalkenyl group may be attached at any heteroatom or carbon atom of the ring such that the result is a stable structure. Heteroycloalkenyl groups of the disclosure can be unsubstituted or substituted. In those embodiments wherein the heterocycloalkenyl group is substituted, the heterocycloalkenyl group can be substituted with 1, 2, or 3 substituents independently selected from D, —OH, —CN, amino, halo, oxo, C₁-C₆alkyl, C₁-C₆alkoxy, C₁-C₆haloalkyl, and C₁-C₆haloalkoxy. Additional substituents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. The term —C₅-C₁₀ heteroaryl refers to a heteroaryl group containing five to ten ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. Heteroaryl groups of the disclosure can be unsubstituted or substituted. In those embodiments wherein the heteroaryl group is substituted, the heteroaryl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substituents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “aryl” when used alone or as part of a substituent group refers to a mono- or bicyclic-aromatic carbon ring structure. Aryl rings can include a total of 6, 7, 8, 9, or 10 ring atoms. Examples of aryl groups include but are not limited to, phenyl, napthyl, and the like. Aryl groups of the disclosure can be unsubstituted or substituted. In those embodiments wherein the aryl group is substituted, the aryl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substituents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “alkenyl” refers to C₂-C₁₂ alkyl group that contains at least one carbon-carbon double bond. In some embodiments, the alkenyl group is optionally substituted. In some embodiments, the alkenyl group is a C₂-C₆ alkenyl.

The term “alkynyl” refers to C₂-C₁₂ alkyl group that contains at least one carbon-carbon triple bond. In some embodiments, the alkenyl group is optionally substituted. In some embodiments, the alkynyl group is a C₂-C₆ alkynyl.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

When a range of carbon atoms is used herein, for example, C₁-C₆, all ranges, as well as individual numbers of carbon atoms are encompassed, for example, “C₁₋₃” includes C₁₋₃, C₁₋₂, C₂₋₃, C₁, C₂, and C₃. The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond.

The term “C₀-C₆alk” when used alone or as part of a substituent group refers to an aliphatic linker having 0, 1, 2, 3, 4, 5 or 6 carbon atoms. The term “—C₁alk-”, for example, refers to a —CH₂—. The term “—C₀alk-” refers to a bond.

Moieties of the disclosure, for example, —C₁-C₆alkyl, —C₁-C₁₀ alkyl, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalky, are optionally substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substituents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Geometric isomers of the compounds of the present invention are described and may be isolated as a mixture of isomers or as separated isomeric forms.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

Compounds of the invention may also include tautomeric forms. All tautomeric forms are encompassed.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

In some embodiments, the compounds of the invention, and salts thereof, are substantially isolated. By “substantially isolated” is meant that the compound is at least partially or substantially separated from the environment in which was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes mammals, and in particular, humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compound 1, compound 2, compound 3, compound 4, compound 5, compound 6 and compound 7 as described herein, as well as any stereoisomers (e.g., enantiomers, diastereomers) and constitutional isomers (e.g., tautomers) of compound 1, compound 2, compound 3, compound 4, compound 5, compound 6 and compound 7 as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

Compounds of the invention can also include all isotopes of atoms occurring in the intermediates or final compounds. Isotopes include those atoms having the same atomic number but different mass numbers. For example, isotopes of hydrogen include tritium and deuterium.

The disclosure is directed to compounds of compound 1, compound 2, compound 3, compound 4, compound 5, compound 6 or compound 7:

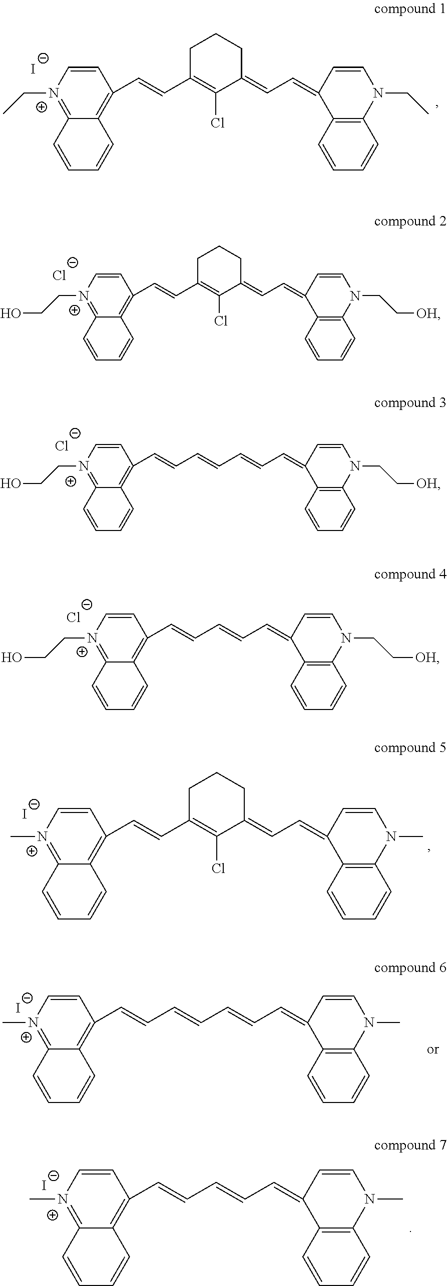

Also provided is a method, comprising treating a biological tissue with a compound according to the present disclosure.

Further provided is a method, comprising synthesizing a compound according to the present disclosure.

In some embodiments, the disclosure is directed to compound 1:

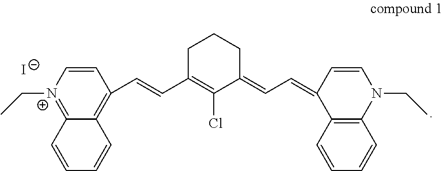 [SMILES: CCN1C=C/C(=C/C=C2\CCCC(/C=C/c3cc[n+](CC)c4ccccc34)=C2Cl)c2ccccc21 ; [I-]]

In some embodiments, compound 1 is referred to as JAM317.

In some embodiments, the disclosure is directed to compounds of compound 2:

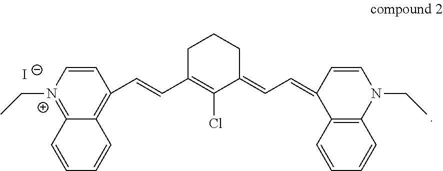 [SMILES: CCN1C=C/C(=C/C=C2\CCCC(/C=C/c3cc[n+](CC)c4ccccc34)=C2Cl)c2ccccc21 ; [I-]]

In some embodiments, compound 2 is referred to as JAM318.

In some embodiments, the disclosure is directed to compounds of compound 3:

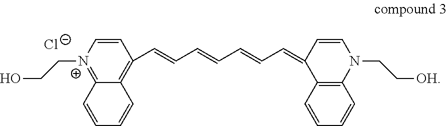 [SMILES: OCCN1C=C/C(=C/C=C/C=C/C=C/c2cc[n+](CCO)c3ccccc23)c2ccccc21 ; [Cl-]]

In some embodiments, compound 3 is referred to as JAM319.

In some embodiments, the disclosure is directed to compounds of compound 4:

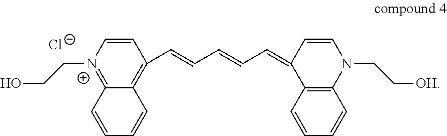 [SMILES: OCCN1C=C/C(=C/C=C/C=C/c2cc[n+](CCO)c3ccccc23)c2ccccc21 ; [Cl-]]

In some embodiments, compound 4 is referred to as JAM320.

In some embodiments, the disclosure is directed to compounds of compound 5:

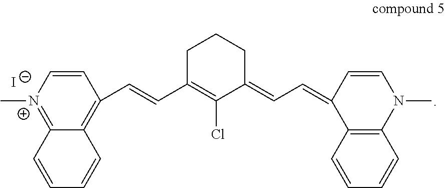

In some embodiments, the disclosure is directed to compounds of compound 6:

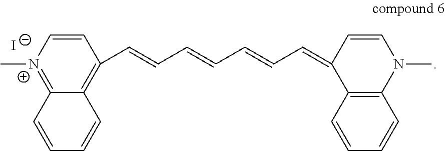

In some embodiments, the disclosure is directed to compounds of compound 7:

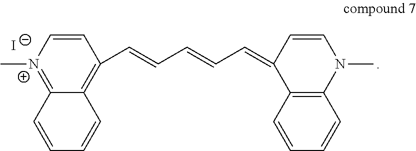

In some embodiments, the disclosure is directed to a pharmaceutical composition comprising compound 1, compound 2, compound 3, compound 4, compound 5, compound 6 or compound 7.

In some embodiments, the disclosure is directed a method of imaging cancer or an inflammatory disease, the method comprising:
contracting tissue with a compound of claim 1 or a pharmaceutical composition comprising the compound;
illuminating the tissue with an excitation light of a wavelength absorbed by the compound; and
detecting an optical signal emitted by the compound in the tissue.

In some embodiments, the tissue is biological tissue. In some embodiments, the tissue is in a human or an animal.

In some embodiments, the method comprises:
illuminating the tissue comprises illuminating a surface of the tissue with the excitation light; and
detecting the optical signal comprises detecting fluorescence from the compound.

In some embodiments, the method comprises:
filtering the light detected by a fluorescence detector to separate out fluorescent components; and
forming an image of the tissue surface.

In some embodiments, the excitation light is continuous wave (CW) in nature.

In some embodiments, the method is used to assist in detection, diagnosis, surgery, staging, treatment, monitoring of treatment, monitoring of disease progression or monitoring therapy.

In some embodiments, the method is used to assist in detection of a disease or disorder.

In other embodiments, the method is used to assist in diagnosis of a disease or disorder.

In other embodiments, the method is used to assist in surgery.

In other embodiments, the method is used to assist in staging.

In yet other embodiments, the method is used to assist in treatment of a disease or disorder.

In yet other embodiments, the method is used to assist in monitoring of treatment.

In yet other embodiments, the method is used to assist in monitoring of disease progression.

In yet other embodiments, the method is used to assist in monitoring therapy.

In some embodiments, the method is used to assist in detection, diagnosis, surgery, staging, treatment, monitoring of treatment, monitoring of disease progression or monitoring therapy of cancer or of a precancerous condition.

In some embodiments, the method is used to assist in detection of cancer or of a precancerous condition.

In other embodiments, the method is used to assist in diagnosis of cancer or of a precancerous condition.

In other embodiments, the method is used to assist in surgery.

In other embodiments, the method is used to assist in staging.

In yet other embodiments, the method is used to assist in treatment of cancer or of a precancerous condition.

In yet other embodiments, the method is used to assist in monitoring of cancer treatment.

In yet other embodiments, the method is used to assist in monitoring of cancer progression.

In yet other embodiments, the method is used to assist in monitoring cancer therapy.

In some embodiments, the cancer is colorectal cancer, esophageal cancer, breast cancer, prostate cancer, head cancer, neck cancer, ovarian cancer, rectal cancer, pancreatic cancer, thyroid cancer, gastric cancer or a sarcoma.

In some embodiments, the cancer is colorectal cancer. In some embodiments, the cancer is esophageal cancer. In some embodiments, the cancer is breast cancer. In some embodiments, the cancer is prostate cancer. In other embodiments, the cancer is head cancer. In other embodiments, the cancer is neck cancer. In other embodiments, the cancer is ovarian cancer. In yet other embodiments, the cancer is rectal cancer. In yet other embodiments, the cancer is pancreatic cancer. In yet other embodiments, the cancer is thyroid cancer. In yet other embodiments, the cancer is gastric cancer. In yet other embodiments, the cancer is a sarcoma.

In some embodiments, the compound used in the methods described herein is compound 1 or JAM317.

In some embodiments, the compound used in the methods described herein is compound 2 or JAM318.

In some embodiments, the compound used in the methods described herein is compound 3 or JAM319.

In some embodiments, the compound used in the methods described herein is compound 4 or JAM320.

In some embodiments, the compound used in the methods described herein is compound 5.

In some embodiments, the compound used in the methods described herein is compound 6.

In some embodiments, the compound used in the methods described herein is compound 7.

The compounds described herein are advantageously used as fluorophores for the following reasons:
1. The compounds described herein absorb and emit in the NIR-II window, which has improved resolution and increased light penetration through tissue in vivo compared to currently available fluorophores in the NIR-I window (700-900 nm). Decreased tissue scattering, absorption, and autofluorescence in the NIR-II range improves resolution in vivo.
2. Compared to known NIR-II fluorophores, the compounds described herein have a low molecular weight, which eases their translation into the clinical space.
3. The core structure of the compounds described herein is easily modifiable at the nitrogen in the head group and at the chlorine-substituted carbon in the polymethine linker region. Targeting groups can be introduced to these locations to target the fluorophores to different disease states or for imaging specific biology in vivo. Further decreased tissue scattering, absorption, and autofluorescence in the NIR-II range improves resolution in vivo.
4. The compounds described herein are able to penetrate deeper into tissue, with strong emission observed up to 3 mm deep as compared to existing NIR probes JAS239 and ICG.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### Intermediate 1: 1-(2-hydroxyethyl)-4-methylquinolin-1-ium chloride

4-methylquinline (lepidine), 2-chloroethan-1-ol, toluene, and ethanol were obtained from Millipore Sigma. 17 mL (18,411 mg, 128.58 mmol) of lepidine were mixed with 18 mL (21,618 mg, 268.51 mmol) of 2-chloroethan-1-ol. The solution was heated and stirred at 130 °C in a 45 mLParr autoclave with a magnetic stirring bar for 2 hrs. The resulting viscous, brown liquid was poured into hot toluene while stirring. A brownish precipitate was obtained and recrystallized twice by dissolving in 100 mL ethanol and pouring into 700 mL of toluene resulting in a white powder following lyophilization.

### Intermediate 2: 1,1′-(2-chlorocyclohex-1-ene-1,3-diyl)bis(N-phenylmethanimine)

Dimethylformamide (DMF), phosphoryl chloride (POCb), cyclohexanone, hydrochloric acid (HCl), and aniline were purchased from Millipore Sigma. Dichloro-methane (DCM) and methanol were obtained from ThermoFisher Scientific. 20 mL of anhydrous DMF and 20 mL of DCM were mixed on ice. 18.5 mL (198 mmol) of POCb mixed in 20 mL of DCM were added to the DMF-DCM solution dropwise under argon over 3 hrs. 5 g (5.275 mL, 50.94 mmol) of cyclohexanone were added to the solution and stirred on ice for 30 min resulting in a bright yellow solution. The reaction mixture was then refluxed at 60 °C for 2 hrs before increasing the temperature to 65 °C and 70 °C for 30 min and 10 min, respectively. The solution was allowed to rest for 10 min before pouring the mixture onto ice to crystallize. The resulting precipitate was filtered to isolate a yellow powder and dry by lyophilization for 2 hrs. 3.95 g (22.88 mmol) of the resulting aldehyde were dissolved in 30.5 mL of DMF and 50 mL of ethanol. The solution was cooled to −20′C in a methanol-liquid nitrogen bath. 17 mL of 12.1 M HCl were added slowly by pipet while stirring. The resulting solution was a viscous orange liquid. The reaction mixture was warmed to 0 °C in an ice water bath. 6.3 mL (69.14 mmol) of aniline were added by pipet with slow stirring for 30 min. The solution turned to a dark violet and was poured onto ice to precipitate. The metallic purple solid was isolated by vacuum filtration and rinsed with ice-cold H₂O before drying overnight by lyophilization.

### Example 1: 4-((E)-2((E)-2-chloro-3-(2-((Z)-J-ethylquinolin-4(JH)-ylidene)-ethylidene) cyclohex-1-en-1-yl)vinyl)-J-ethylquinolin-1-ium iodide (compound 1/JAM317)

1-ethyl-4-methylquinolin-1-ium iodide, anhydrous pyridine, and anhydrous triethylamine were purchased from Millipore Sigma. Dimethyl sulfoxide (DMSO) was obtained from ThermoFisher Scientific. 2.00 g (6.195 mmol) of Intermediate 2 were mixed with 3.33 g (11.13 mmol) of 1-ethyl-4-methylquinolin-1-ium iodide in 46 mL of anhydrous pyridine. 4 mL of anhydrous triethylamine were added slowly by syringe. The solution changed color gradually from a deep red to blue. The solution was swirled and left to rest overnight.

Precipitation was facilitated by placing the reaction mixture in a −20 °C freezer overnight. The precipitate was isolated by vacuum filtration and dried by lyophilization. Purification was performed by precipitation of 1.44 g product dissolved in 25 mL of DMSO before pouring into 1 L of ice-cold H₂0. The mixture was left to settle for 1 hour before isolating the precipitate by vacuum filtration and drying overnight by lyophilization.

### Example 2: 4-((E)-2((E)-2-chloro-3-(2-((Z)-1-hydroxyethylquinolin-4(JH)-ylidene)-ethylidene)cyclohex-1-en-1-yl)vinyl)-1-hydroxyethylquinolin-1-ium chloride (compound 2/JAM318)

0.762 g (2.12 mmol) of (2) were mixed with 0.948 g (4.24 mmol) of Intermediate 1 in 18 mL of anhydrous pyridine. 1.5 mL of anhydrous triethylamine were added slowly by syringe. The solution changed color gradually from a deep red to blue. The solution was swirled and left to rest overnight. Precipitation was facilitated by placing the reaction mixture in a −20 °C freezer overnight. The precipitate was isolated by vacuum filtration and dried by lyophilization. Purification was performed by precipitation of product dissolved in DMSO before pouring into ice-cold H₂0 (1:40, v:v). The mixture was left to settle for 1 hour before isolating the precipitate by vacuum filtration and drying overnight by lyophilization.

### Example 3: l-(2-hydroxyethyl)-4-((JE,3E,5E)-7-((Z)-J-(2-hydroxyethyl)-quinoline-4(JH)-ylidene)hepta-1,3,5-trien-1-yl)quinoline-1-ium chloride (compound 3/JAM319)

1.273 g (4.47 mmol) of (1E,2E,5E)-N1,N⁵-diphenylpent-2-ene-1,5-diimiine were mixed with 2.0 g (8.94 mmol) of Intermediate I in 37 mL of anhydrous pyridine. 3.2 mL of anhydrous triethylamine were added slowly by syringe. The solution changed color gradually from a deep red to blue. The solution was swirled and left to rest overnight. Precipitation was facilitated by placing the reaction mixture in a −20 °C freezer overnight. The precipitate was isolated by vacuum filtration and dried by lyophilization. Purification was performed by precipitation of product dissolved in DMSO before pouring into ice-cold H₂O (1:40, v:v). The mixture was left to settle for 1 hour before isolating the precipitate by vacuum filtration and drying overnight by lyophilization.

### Example 4: l-(2-hydroxyethyl)-4-((JE,3E)-5-((Z)-J-(2-hydroxyethyl)quinolin-4(JH)-ylidene)penta-1,3-dien-4-yl)quinolin-1-ium chloride (compound 4/JAM320)

1.157 g (4.47 mmol) of (1E,3E)-N1,N³-diphenylpropane-1,3-diimine were mixed with 2.0 g (8.94 mmol) of Intermediate 1 in 37 mL of anhydrous pyridine. 3.2 mL of anhydrous triethylamine were added slowly by syringe. The solution changed color gradually from a deep red to blue. The solution was swirled and left to rest overnight. Precipitation was facilitated by placing the reaction mixture in a −20 °C freezer overnight. The precipitate was isolated by vacuum filtration and dried by lyophilization. Purification was performed by precipitation of product dissolved in DMSO before pouring into ice-cold H₂O (1:40, v:v). The mixture was left to settle for 1 hour before isolating the precipitate by vacuum filtration and drying overnight by lyophilization.

### Example A—In Vitro Studies

### Absorbance and Emission Spectra

Absorbance spectra. The absorbances for compound 1 and compound 2 were obtained using a PerkinElmer Lambda 5 measuring absorbances in 1 nm steps from 500 to 1100 nm. The absorbance spectrum for compound 3 was obtained using a JASCO VI 70 measuring in 1 nm steps from 850 to 1150 nm. All compounds were dissolved in DMSO at a concentration of 5 μM.

Emission spectra. The emissions for compounds 1, 2 and 3 were obtained using a Horiba QuantaMaster with excitation at 900 nm and emissions recorded in 1 nm steps from 925 to 1300 nm.

NIR-II Fluorescence of Compound 1 vs. JAS239 and ICG.

Compound 1, JAS239, and ICG were dissolved in DMSO and added to a 96-well black-well clear-bottom plate at concentrations from 1 μM to 100 μM. The plate was then imaged using the PhotonEtc IR Vivo using the NIR-II emission filter (1000-1250 nm) with excitation at 760 nm, 808 nm, 890 nm, or 940 nm.

Depth Penetration Comparisons of Compound 1 vs. JAS239 and ICG.

Compound 1, JAS239, and ICG were diluted in DMSO and placed in a 96-well black-well clear-bottom plate at concentrations from 0.1 μM to 100 μM. The plate was then imaged using the PhotonEtc IR Vivo using the NIR-II emission filter with excitation at 760, 808, 890, or 940 nm. Wells were then covered with thinly sliced raw chicken breast tissue from 1 mm to 3 mm thick. Imaging was then repeated for each tissue thickness.

### Cell Uptake.

KLN 205 cells were seeded into an 8 chamber slide at 15,000 cells/well. Cells were incubated overnight at 37 °C and 5% CO2. Cells were then treated in 500 μL with 10 μM of compound 1, compound 2 or compound 3, or with vehicle (DMSO). The slide was incubated for 2 hours with treatment at 37 °C and 5% CO₂.

Following treatment, the solution was aspirated, and cells were rinsed twice with 500 μL of ice-cold DPBS. Triton X-100 was diluted to be 0.5% Triton, and 500 μL of this solution were added to each chamber for 10 min at 37 °C and 5% CO2 to lyse the cells. 100 μL were removed from each chamber and added to a 96-well black-well clear-bottom plate. The plate was then imaged for NIR-II emission using the PhotonEtc IR Vivo with excitation at 760, 808, 890, or 940 nm.

### Results

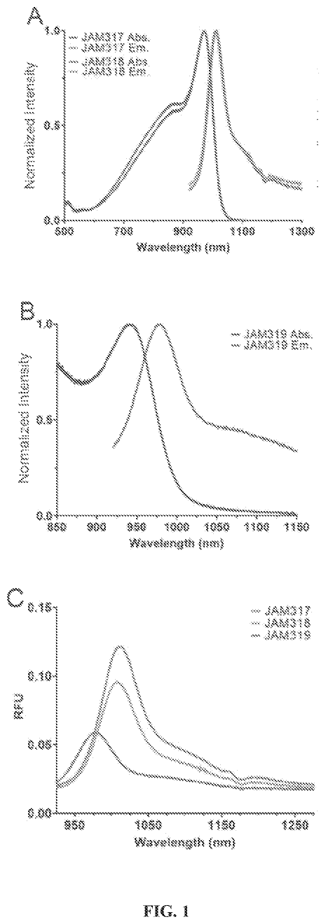

Synthesis of compound 1, compound 2 and compound 3 was verified by MALDI-TOF mass spectrometry with further confirmation by ¹H-NMR. Absorbance and emission spectra for compound 1, compound 2 and compound 3 were measured at 5 μMin DMSO, as shown in FIGS. 1A, 1B and 1C. The absorbance and emission maxima for compound 1 and compound 2 were further shifted into the NIR-II window compared to compound 3. Compound 1 exhibited the most intense emission.

As seen in FIG. 1, compound 1 and compound 2 have similar absorbance and emission spectra, with a maximum absorbances at 970 nm and maximum emissions at 1010 and 1007 nm, respectively. Compound 3 has a maximum absorbance at 942 nm and maximum emission at 979 nm. The emission intensities were determined with excitation at 900 nm.

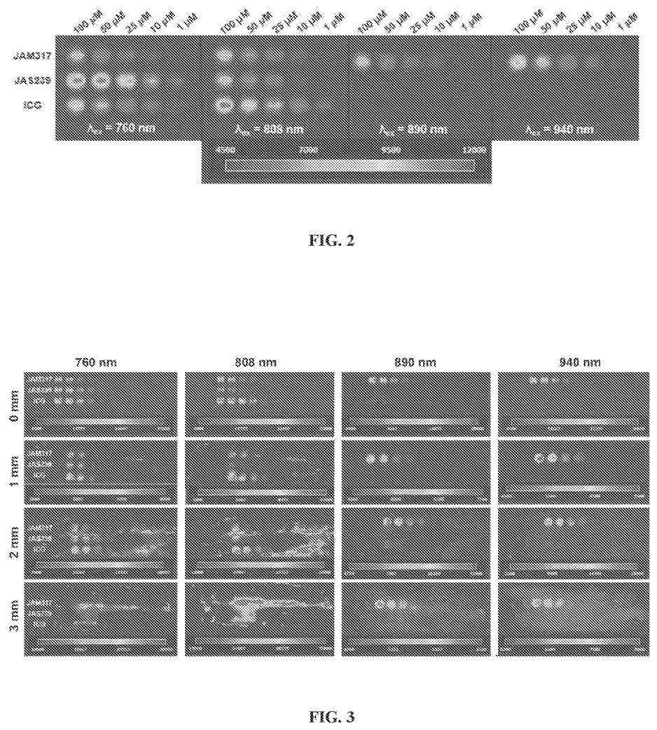

NIR-II emission for compound 1 was compared to the well-characterized indocarbocyanines, JAS239 and ICG, as shown in FIG. 2. Dyes were excited with excitation lasers at 760, 808, 890, and 940 nm and emissions were measured between 1,000 and 1,250 nm. All dyes exhibited NIR-II fluorescence. However, only compound 1 had measurable fluorescence in the NIR-II window with 890 and 940 nm excitation lasers. With shorter wavelength excitation lasers (760 and 808 nm) JAS239 and ICG exhibited the strongest NIR-II emissions. When excited by the longer wavelength 890 and 940 nm lasers, JAS239 and ICG NIR-II emissions disappeared, and compound 1 demonstrated a strong NIR-II signal.

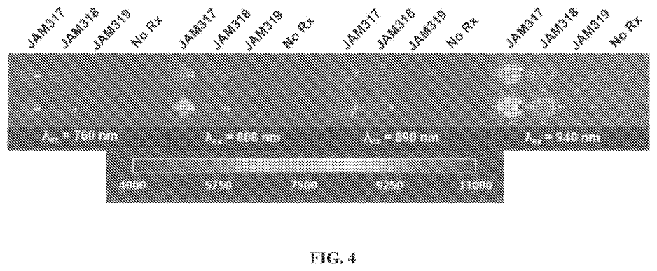

Resolution of compound 1's fluorescence was then compared to JAS239 and ICG fluorescence with increasing tissue phantom depth, as shown in FIG. 3. ICG exhibited the most intense emissions in the NIR-II window when no tissue was present, but emission and resolution of ICG was ablated with increasing tissue depth. Compound 1 exhibited strong emission with resolvable signal at tissue depths up to 3 mm. NIR-II signal decreased with increasing tissue thickness. However, when exciting with longer wavelength lasers (890 and 940 run), the fluorescence of compound 1 remained well-resolved and exhibited high tissue penetration with low autofluorescence even at 3 mm tissue depth.

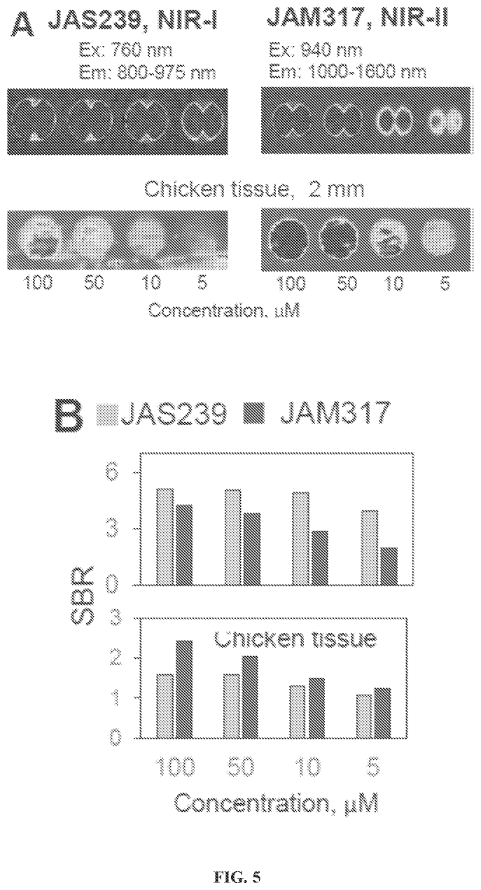

Cell uptake of compound 1, compound 2 and compound 3 was evaluated by incubating KLN 205 cells in 10 μM of compound 1, compound 2 and compound 3, or with DMSO for 2 hrs. Following incubation, cells were rinsed twice with ice-cold DPBS before lysing by treatment with Triton. The lysates were then transferred to a 96-well black-well clear-bottom plate and imaged for NIR-II fluorescence in the IR Vivo, as shown in FIG. 4. Lysates from cells treated with compound 1 and compound 2 had measurable emissions, but no fluorescence was observed in lysates for cells treated with compound 3 or DMSO.

Imaging of ChoKα NIR-I probe (JAS239) and ChoKα NIR-II probe (compound 1) using tissue phantom is shown in FIG. 5. In FIG. 5A, the plate was uncovered (left panel) or covered by 2 mm chicken tissue (right panel). Imaging of the probes dilution with and without plate covering by the tissue. In FIG. 5B, SBR was shown for each fluorescent spot of the images.

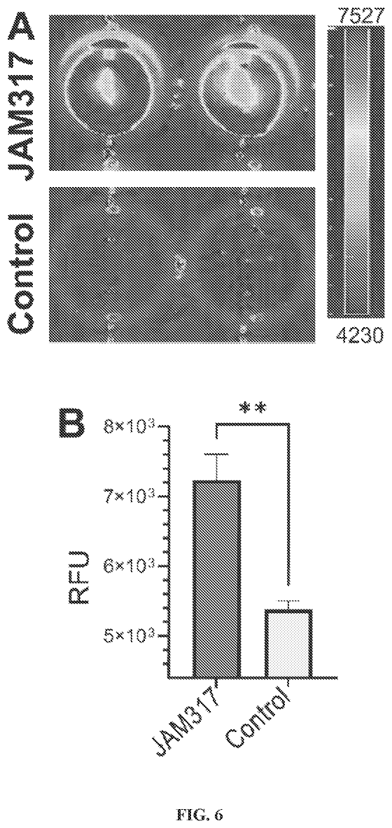

Intracellular uptake and retention of ChoKα-targeted NIR-II probe by KLN-205 cancer cells (1.5×104 cells total), measured with IR VIVO, is shown in FIG. 6.

### DISCUSSION

Compound 1, compound 2 and compound 3 were designed to be modifiable with an extended electron-conjugated region compared to the indocarbocyanines. The central chlorine attached to the polymethine linker was designed to allow for the substitution of targeting moieties for biomarkers of NSCLC. The absorbance and emission spectra of compound 1, compound 2 and compound 3 demonstrated measurable NIR-II fluorescence making them promising candidates for further investigation into targeting for NSCLC imaging. Compound 1 exhibited the highest fluorescence with an emission maximum in the NIR-II window making it the lead fluorophore for future studies.

Few NIR-II imaging systems have been developed with limited market availability. The PhotonEtc IR Vivo was installed in the University of Pennsylvania Small Animal Imaging Facility in February 2022. Initial testing of JAM317 on the IR Vivo confirmed NIR-II fluorescence of the probe and showed comparable NIR-II emissions to ICG when excited near its absorbance maximum.

The primary goal of developing the NIR-II fluorophores was to improve on the in vivo resolution observed for NIR-I fluorophores. ICG and JAS239 have measurable emissions in the NIR-II window, but both probes require excitation with a NIR-I laser. Thus, compound 1 was tested for NIR-II fluorescence emission intensity and resolution at increasing tissue depths compared to ICG and JAS239 with excitation from 760 to 940 nm using four different lasers.

Chicken breast tissue was layered over wells containing varying concentrations of each fluorophore in 1 mm sections. Fluorescence in the NIR-II window was measured following excitation at 760, 808, 890, and 940 nm. At 3 mm tissue depth, none of the probes exhibited resolvable fluorescence when excited with a NIR-1 laser. However, wells containing compound 1 were easily resolved at 3 mm when using longer wavelength excitation lasers. Tissue scattering, absorption, and autofluorescence decreases at increasing wavelengths of light. Thus, the improved resolution of compound 1 with increasing wavelength of excitation laser likely resulted from improved tissue penetration of the excitation laser. Compound 1 has a maximum absorption at 970 nm resulting in strong excitation of the probe and fluorescence conversion.

Compound 1, compound 2 and compound 3 were tested for cell uptake by KLN 205 cells. To target intracellular cancer biomarkers such as ChoKa, the probes would need to be internalized by cells. Compound 1, compound 2 and compound 3 were incubated with KLN 205 cells, and the resulting lysates were tested for NIR-II fluorescence following rinsing to determine if compound 1, compound 2 or compound 3 can partition into cells. Lysates from cells treated with compound 1 and compound 2 exhibited measurable fluorescence in the NIR-II window demonstrating intracellular localization of the probes prior to cell lysis.

Compound 1, compound 2 and compound 3 represent a new family of NIR-II fluorophores with low molecular weights. Compound 1 exhibited improved fluorescence resolution compared to well-established fluorophores when imaged through chicken breast tissue making them promising candidates in development for in vivo imaging. The easily modifiable synthetic scheme promotes the introduction of targeting moieties to various diseases including NSCLC.

### Example B—In Vivo Studies

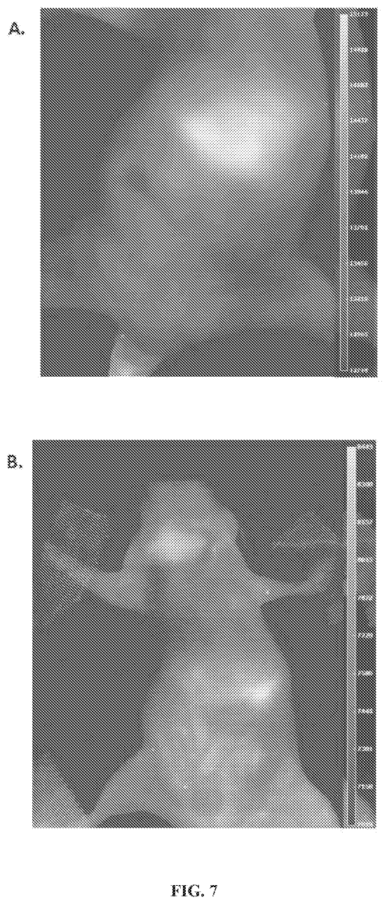

Athymic Nu/Nu mice were injected with 40 nmol compound 1 through the tail vein in Normal Saline and filtered through a 0.22 μm filter. The results are shown in FIG. 7. A ventral image of mice at 5 mins after injection shows compound 1 in the liver, as shown in FIG. 7A. A ventral image of mice at 2 hours after injection shows compound 1 in the throat, liver and intestines, as shown in FIG. 7B.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound having the structure of compound 1, compound 2, compound 3, compound 4, compound 5, compound 6 or compound 7:

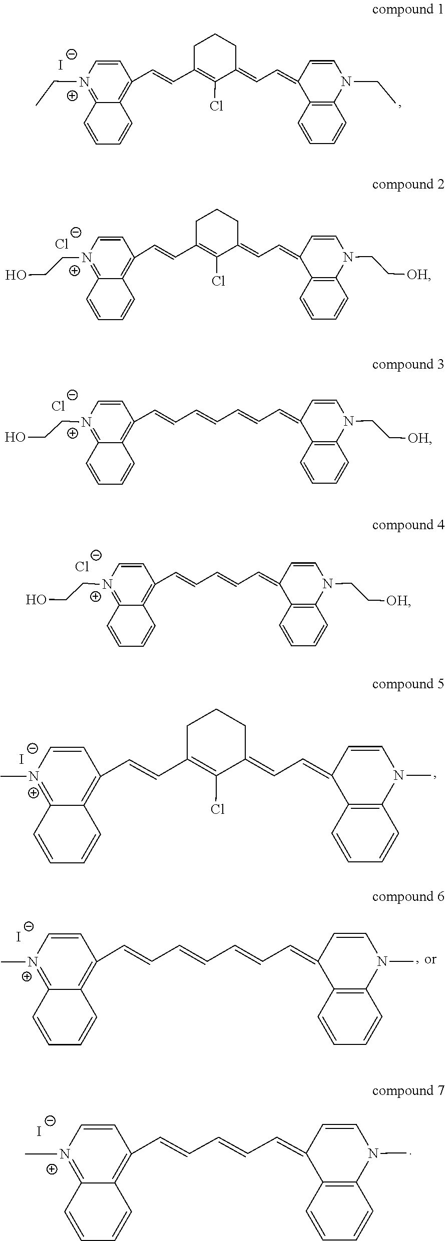

2. The compound of claim 1, wherein the compound is:

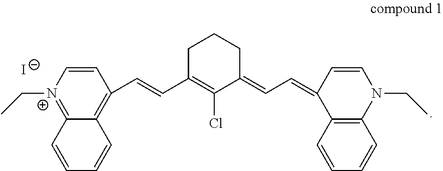 [SMILES: CCN1C=C/C(=C/C=C2\CCCC(/C=C/c3cc[n+](CC)c4ccccc34)=C2Cl)c2ccccc21 ; [I-]]

3. The compound of claim 1, wherein the compound is:

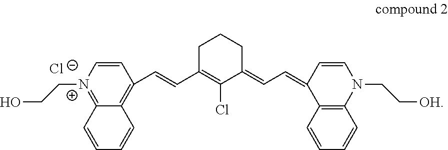 [SMILES: OCCN1C=C/C(=C/C=C2\CCCC(/C=C/c3cc[n+](CCO)c4ccccc34)=C2Cl)c2ccccc21 ; [Cl-]]

4. The compound of claim 1, wherein the compound is:

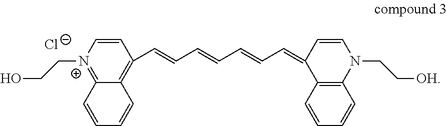 [SMILES: OCCN1C=C/C(=C/C=C/C=C/C=C/c2cc[n+](CCO)c3ccccc23)c2ccccc21 ; [Cl-]]

5. The compound of any claim 1, wherein the compound is:

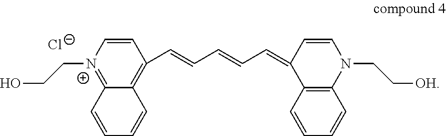 [SMILES: OCCN1C=C/C(=C/C=C/C=C/c2cc[n+](CCO)c3ccccc23)c2ccccc21 ; [Cl-]]

6. The compound of claim 1, wherein the compound is:

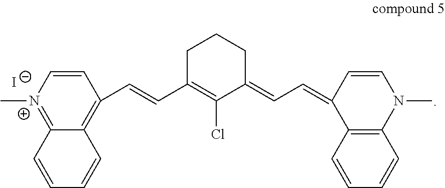

7. The compound of claim 1, wherein the compound is:

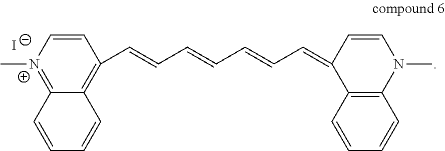

8. The compound of claim 1, wherein the compound is:

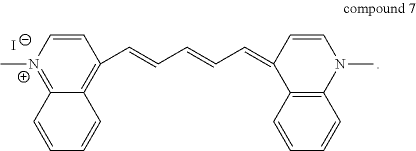

9. A pharmaceutical composition comprising a compound of claim 1.

10. A method of imaging cancer or an inflammatory disease, the method comprising:
contracting tissue with a compound of claim 1 or a pharmaceutical composition comprising the compound;
illuminating the tissue with an excitation light of a wavelength absorbed by the compound; and
detecting an optical signal emitted by the compound in the tissue.

11. The method of claim 10, wherein the tissue is biological tissue.

12. The method of claim 10, wherein the tissue is in a human or an animal.

13. The method of claim 10, wherein:
illuminating the tissue comprises illuminating a surface of the tissue with the excitation light; and
detecting the optical signal comprises detecting fluorescence from the compound.

14. The method of claim 10, further comprising:
filtering the light detected by a fluorescence detector to separate out fluorescent components; and
forming an image of the tissue surface.

15. The method of claim 10, wherein the excitation light is continuous wave (CW) in nature.

16. The method of claim 10, wherein the method is used to assist in detection, diagnosis, surgery, staging, treatment, monitoring of treatment, monitoring of disease progression or monitoring therapy.

17. The method of claim 10, wherein the method is used to assist in detection, diagnosis, surgery, staging, treatment, monitoring of treatment, monitoring of disease progression or monitoring therapy of cancer or of a precancerous condition.

18. The method of claim 17, wherein the cancer is colorectal cancer, oesophageal cancer, breast cancer, prostate cancer, head cancer, neck cancer, ovarian cancer, rectal cancer, pancreatic cancer, thyroid cancer, gastric cancer or a sarcoma.

19. The method of claim 10, wherein the compound is:

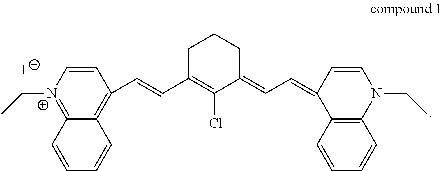 [SMILES: CCN1C=C/C(=C/C=C2\CCCC(/C=C/c3cc[n+](CC)c4ccccc34)=C2Cl)c2ccccc21 ; [I-]]

20. The method of claim 10, wherein the compound is:

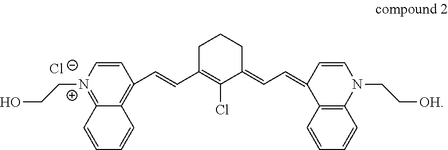 [SMILES: OCCN1C=C/C(=C/C=C2\CCCC(/C=C/c3cc[n+](CCO)c4ccccc34)=C2Cl)c2ccccc21 ; [Cl-]]

21. The method of claim 10, wherein the compound is:

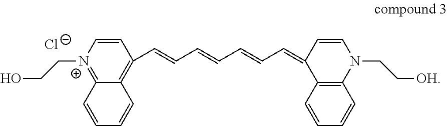 [SMILES: OCCN1C=C/C(=C/C=C/C=C/C=C/c2cc[n+](CCO)c3ccccc23)c2ccccc21 ; [Cl-]]

22. The method of claim 10, wherein the compound is:

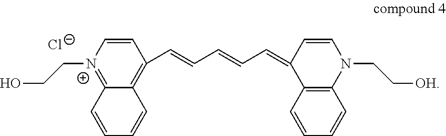 [SMILES: OCCN1C=C/C(=C/C=C/C=C/c2cc[n+](CCO)c3ccccc23)c2ccccc21 ; [Cl-]]

23. The method of claim 10, wherein the compound is:

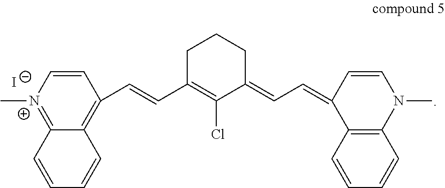

24. The method of claim 10, wherein the compound is:

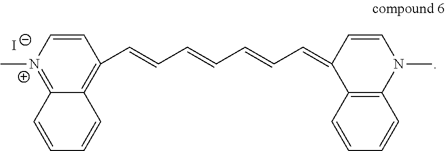

25. The method of claim 10, wherein the compound is: [chemical structure]
